(19) United States
(12) Patent Application Publication
(10) Pub. No.: US 2007/0172433 A1
(43) Pub. Date: Jul. 26, 2007

(54) Biometic compounds containing hydroxyapatites substituted with magnesium and carbonate, and the processes used to obtain them

(76) Inventor: Norberto Roveri, Bologna (IT)

Correspondence Address: Jay S. Cinamon;Abelman, Frayne and Schwab, 666 Third Avenue, New York, NY, 10017-5621, US

(21) Appl. No.: 10/590,186

(22) PCT Filed: Feb. 25, 2005
(86) PCT No.: PCT/EP05/50815

§ 371 Date: Aug. 18, 2006

(30) Foreign Application Priority Data
Feb. 27, 2004 (IT) FI2004A000048

(51) Int. Cl. A61K 33/42; A61K 31/734; A61K 8/73

(52) U.S. Cl. 424/49; 424/601; 514/54

(57) ABSTRACT

Carbonate hydroxyapatites containing magnesium, composite materials consisting of said hydroxyapatites and natural polymers are described; processes for the preparation of said materials and their use in orthopaedics and dentistry fields are also described.

### FIELD OF THE INVENTION

The present invention concerns materials that are useful for the treatment of bone defects in the fields of orthopaedics and dentistry.

### BACKGROUND OF THE INVENTION

For a long time, the inorganic phase of bones and teeth has been represented and idealized as stoichiometric hydroxyapatite (HA: Ca₁₀(PO₄)₆(OH)₂). During the past years, research has focussed on the synthesis of non-stoichiometric hydroxyapatite containing specific substituting ions, since in reality the major component of biological tissue is hydroxyapatite that is variously substituted at both the cationic and anionic reticular sites. Among the substituents of the calcium ion (Ca²⁺), Mg²⁺ is of particular interest, as it plays an important part in the development of new bone tissue. It has been shown that in calcified tissues the quantity of Mg present in the apatite phase is greatest at the beginning of the calcification process and decreases as the mineralization progresses. Moreover, it appears that Mg might play an important role in the qualitative alterations to the bone matrix, whose fragility is determined by these changes. A shortage of Mg has a negative effect on all stages of the skeletal metabolism, as it causes the cessation of bone growth, the reduction of the activity of osteoblasts and osteoclasts, osteopaenia and bone fragility.

Studies of the chemical synthesis of partially Mg-substituted apatite have shown that in solution this ion inhibits the crystallization of the apatite, resulting in a synthetic apatite with a low level of crystallinity, which makes it even morphologically more similar to natural apatite. At the same time, the synthetic Mg-hydroxyapatite is more soluble and thus more absorbable than non-substituted HA. However, there is a limit to the incorporation of Mg into apatite, as high concentrations of this ion tend to destabilize the apatite's structure. Molar ratios above 0.3 of Mg/Ca in solution proportionately increase the probability of the formation of tricalcium magnesium phosphate to the detriment of Mg-substituted HA. The substitution of the Ca ion with Mg can be increased by simultaneously incorporating carbonate ions into the apatite structure. This is of great interest, as the carbonate ion is also found in the structure of natural apatite; its incorporation into synthetic hydroxyapatite must thus be considered of primary importance. The carbonate ion can occupy two different sites in the apatite structure: it can partially substitute the OH⁻ ion (site A) and/or the PO₄³⁻ ion (site B). Both the total carbonate content (in the range of 3-8 wt. %) and the relative quantities of type A and type B carbonation (A/B in the range of 0.7-0.9) found in biological carbonate depend on the age of the individual. Synthetic carbonation should preferably take place at site B, as this results in a reduction of the crystallinity and an increase of the solubility of the apatite phase. Moreover, type A carbonation is characterized by a lesser affinity of the apatite for the osteoblast cells, thus resulting in a lesser cellular adhesion and a decreased production of collagen compared to non-substituted HA.

Thus the synthesis of Mg carbonate hydroxyapatite (MgCHA) is of primary importance in the context of the development of synthetic materials that mimic the inorganic phase of bone tissue both with respect to its composition and to its morphology.

All of this has been well described in scientific literature; see for example
Landi, E., Celotti, G., Logroscino, G. and Tampieri, A., Carbonated Hydroxyapatite as Bone Substitute. J. Eur. Ceram. Soc., 2003, 23, 2931-2937.
Redey, S. A., Razzouk, S., Rey, C., Bemache-Assollant, D., Leroy, G., Nardin, M., and Cournot, G., Osteoclast adhesion and activity on synthetic hydroxyapatite, carbonated hydroxyapatite and natural calcium carbonate: relationship to surface energies. J. Biomed. Mater. Res., 1999, 45, 140-147.
Redey, S. A., Nardin, M., Bemache-Assollant, D., Rey, C., Delannoy, P., Sedel, L. and Marie, P. J., Behaviour of human osteoblastic cells on stoichiometric hydroxyapatite and type A carbonate apatite: role of surface energy. J. Biomed. Mater. Res., 2000, 50, 353-364.
Gibson, I. R., and Bonfield, W., Preparation and Characterization of Magnesium/Carbonate co-substituted Hydroxyapatites, J. Mat. Sci.: Mat Med., 2002, 13, 685-693.
I. R. Gibson and W. Bonfield: J. Biomed. Mater. Res., vol. 59 (2002) p. 697.
A. Bigi, G. Falini, E. Foresti, M. Gazzano, A. Ripamonti and N. Roveri: J. Inorg. Biochem., vol. 49 (1993) p. 69-78.
S. Baravelli, A. Bigi, A. Ripamonti, E. Foresti and N. Roveri: .Inorg. Biochem., vol. 20 (1984) p. 1-12.
A. Bigi, A. Ripamonti, M. H. J. Koch, G. Cojazzi, G. Pizzuto and N. Roveri: Int. J. Biol. Macromol., (1991), vol. 13, p. 110-114.
A. Bigi, E. Foresti, R. Gregoriani, A. Ripamonti, N. Roveri and J. S. Sha, “The Role of Magnesium on the Structure of Biological Apatites” Calcif. Tissue Inf. (1992) vol. 50, p439-444.
A. Bigi, G. Falini, E. Foresti, M. Gazano, A. Ripamonti and N. Roveri, Acta Cryst. (1996), B52, p. 87-92.

We believe that currently synthesized hydroxyapatites present problems (slow bioabsorption and insufficient activation of the osteoblasts) that can be attributed to the fact that they are stoichiometrically pure, while natural apatites contain such doping ions as carbonate and magnesium. It appears that carbonate hydroxyapatites are absorbed better by the osteoclasts, which is probably due to an increase of the solubility caused by the substitution of phosphate with carbonate. Apatites doped with magnesium have demonstrated a kinetic property of faster osteointegration, probably due to the stimulating effect of magnesium on the growth of the osteoblasts and on the secretion of matrix proteins.

Moreover, in synthetic HA it is important that the magnesium is not simply superficially absorbed or inserted into the crystalline matrix, in order to avoid its massive release only at the beginning.

In WO9932400, the synthesis of a hydroxyapatite substituted with carbonate and magnesium is described: the quantity of the incorporated Mg does not exceed 0.5 wt. % and the carbonate does not exceed 1 wt. %, and for its creation a synthesis method was used in which the carbonate is introduced into the reaction mixture without the help of other undesirable ions such as sodium. Moreover, the determined Ca⁺ Mg/P ratio just barely exceeds the theoretical value for non-substituted hydroxyapatites, 1.67; this indicates that the carbonate primarily substitutes the hydroxide group at site A, largely ignoring site B and thus the phosphate. With the process described in this present invention, the percentage of Mg introduced is considerably greater and the carbonate substitution concerns mainly site B.

In WO03089022, a claim is filed for composite materials consisting of an organic matrix and an inorganic mineral phase. In this material, the inorganic phase is absorbed into the organic matrix in a liquid form; more specifically, the organic matrix generally is collagen.

In U.S. Pat. No. 6,569,489, a process is realized in which hydroxyapatite is directly applied onto a substrate suitable for implantation by immersing the substrate in an aqueous liquid solution containing the salts from which the hydroxyapatite forms.

In JP-A-6245992, a carbonate hydroxyapatite is described, which presents considerable differences to the present invention, the most conspicuous among them being the low Mg substitution level (between 0.05 and 0.5 wt. % with a (Ca⁺ Mg)/P ratio between 1.50 and 1.67).

From the above it is obvious that it is necessary to create new materials containing hydroxyapatites with better characteristics, which become more and more similar to the natural product and yet for safety reasons make it possible to avoid using material of animal origin.

### BRIEF DESCRIPTION OF THE DRAWINGS

FIG. 1: is an X-ray diffraction spectrum which shows the characteristic signs of apatites.

FIG. 2: is an IR spectrum which shows the signals relating to the groups PO₄³⁻, HPO₄²⁻, OH and CO₃²⁻.

FIG. 3 reports the results of the thermogravimetric analysis.

FIG. 4 reports the results of the analysis using x-ray diffractometry and confirms that the inorganic phase was not compromised during the granulation process with alginate.

FIG. 5 reports the results of the infrared spectroscopy which shows the characteristic profile of the magnesium carbonate hydroxyapatite to be dominant;

FIG. 6 reports the result of the thermogravimetric analysis showing the weight decreases relating to the decomposition of both the organic and the inorganic phase.

### DETAILED DESCRIPTION OF THE INVENTION

Surprisingly, it has now been discovered that it is possible to create magnesium-containing carbonated hydroxyapatite (MgCHA) with a low level of crystallinity, suitable for creating a compound material in the form of a freeze-dried granulate suitable for bone implants, particularly in the field of dentistry, consisting of said hydroxyapatite and an organic polymer, preferably an alginate.

Hence, both the MgCHA (with a nanostructure with a low level of crystallinity) and the final composite material in the form of a granulate are a part of the present invention, as are the processes for their preparation.

According to the invention, the synthesis method used for the synthesis of the MgCHA makes it possible to selectively introduce the carbonate into the more favourable positions (site B, in place of the phosphate) and to significantly increase the content of magnesium ions. Indeed, the carbonate can enter the hydroxyapatite's structure at two sites, thus it can replace an OH group (site A) or it can replace a phosphate group (site B). The substitution at site B is generally preferable, because it provides the compound with a greater solubility in a biological environment.

The synthesis method used makes it possible to obtain hydroxyapatites with a carbonate substitution in the range of 4 to 10 wt %, preferably between 6 and 8 wt %, and a distribution between the two sites of between 40 and 45% for site A and consequently 55 to 60% for site B.

With the same process it is possible to uniformly dope the carbonate hydroxyapatite with magnesium, resulting in an Mg concentration of between 5 and 15% (expressed as a molar ratio with respect to calcium), preferably between 6 and 8%; or, expressed as weight percentage, an Mg quantity of between 1.0 and 2.72 wt %, preferably between 1.19 and 1.57 wt %. The introduction of Mg causes a partial collapse of the crystalline structure of the hydroxyapatite, but by means of x-ray analysis it is possible to establish the persistence of the characteristic bands of this type of structure; we can therefore confirm that the resulting product still remains a hydroxyapatite, even though at the nanostructure level it presents a low level of crystallinity (estimated around 40-60%). The crystallinity index can be established using x-ray diffraction analysis, e.g. as described in: Landi, E., Tampieri, A., Celotti, G., Sprio, S., J. Eur. Ceram. Soc. 2000, 20, 2377-2387. The molar ratio of (Ca⁺ Mg)/P in the resulting compound exceeds 1.7 and is thus above the value of 1.67 of non-substituted hydroxyapatites; in fact, this shows that the carbonate substitution has taken place mainly at site B. At any rate, the resulting material is homogenous and for purely descriptive purposes can be represented as an intermediate compound between the following two limit-defining formulas, assuming a constant Mg/Ca ratio:
Ca_{6.4}Mg_{0.5}(HPO₄)_{3.8}(CO₃)_{2.1}(OH)₂
Ca_{6.4}Mg_{0.55}(PO₄)_{0.5}(HPO₄)_{3.5}(CO₃)_{1.7}(CO₃),
where formula 1 would show a compound in which all substitutable sites B have been substituted with carbonate and formula 2 a compound in which all substitutable sites A have been substituted with carbonate. Analogous representations can be made for other compounds with a differing Mg/Ca ratio.

The MgCHA is then appropriately mixed with an organic polymer, which serves a double purpose: it acts as a modulator for the interactions of the apatite with the biological environment and it allows for a good workability of the composite with a view to its medical applications. The organic polymer should preferably be a polysaccharide; in this specific case an alginate was used. Alginates are a family of linear polysaccharides of natural origin (algae); they consist of D-mannuronic acid (M) and L-guluronic acid (G), which depending on the source are present in blocks of similar residues, i.e. of the type MMMMMM or GGGGGG, or strictly alternating, i.e. GMGMGMGM. The proportion of the two can vary in percentage from 20 to 80% for M and consequently 80 to 20% for G. Specifically, commercially available sodium alginate was used, for example B.H. Schilling alginate LF 120M (G/M 40/60), B.H. Schilling alginate LF10/60 (G/M 70/30), Fluka alginate (G/M ca. 70/30).

The composite of the present invention consists of a mixture of MgCHA and alginate in proportions ranging from 50:50 to 80:20, preferably in a proportion of 60:40.

The above composites are preferably obtained in the form of a porous granulate in order to facilitate its conversion into a viscous paste through treatment with aqueous solution.

The particle size (the granulometry) can easily be changed during the production process based on the requirements of the composite's application.

Thus, the composite material has the following characteristics:

Osteoconductivity and osteoinductivity, due to the increase of adhesion and growth of the osteoblasts induced by the magnesium.

Bioabsorption, induced by the favourable changes to the crystallinity (while the specific characteristics of the hydroxyapatite are retained), solubility and granulometry induced by the carbonate and/or magnesium and/or polymer.

Workability due to the jellifying properties of the polymer.

Safety, as there are no materials of animal origin.

Using currently known methods, the described composite material can easily be produced in final forms other than porous granulate; examples are gel, malleable paste, malleable putty, sponge, particulate or pre-formed solid that can be moulded according to the application requirements.

The material may be suitably sterilized by irradiation with gamma radiation.

At the time of use, the required quantity of the composite may be applied after it has been appropriately mixed with an aqueous solution. This solution may for example be chosen from the following group: sterile water, sterile saline solution, phosphate PBS buffer solution, blood or its derivatives (e.g. platelet gel, plasma, etc.), in order to obtain a gelatinous paste that is dense but easily worked.

The hydroxyapatite of the present invention may be obtain through a synthesis process as described below:

A phosphoric acid solution and a sodium bicarbonate solution are added simultaneously over a period of 2-8 hours (preferably 3-5 hours) to a calcium hydroxide suspension and a magnesium salt (preferably hexahydrated magnesium chloride), while the temperature is maintained at 30 to 60° C. (preferably between 35 and 45° C.). Once the addition is complete, the mixture is stirred for 1-6 hours and is then left to rest at room temperature for 10-48 hours (preferably 20-28 hours). The hydroxyapatite is filtered by centrifugation or filtration, is washed with water and is dried.

In a mixer, the MgCHA obtained in this way is mixed with sodium alginate (both in the form of sifted powder) in proportions ranging from 50:50 to 80:20, preferably 60:40. While stirring continuously, an organic solvent, preferably ethyl alcohol or ethyl ether, is added in order to allow for a more thorough amalgamation. The successive removal of the solvent, first quickly in the oven, then by a long period of freeze-drying (24-48 hours), makes it possible to obtain the final granulated composite material.

Similar granulation processes using water or aqueous solvents result in composite with reduced stability. On the other hand, once evaporation at a low temperature is complete, the granulation method using a solvent on the basis of alcohol or ether results in a stable granulate that can be stored at room temperature.

The composite of the present invention may be used for the treatment of patients with a loss of bone substance through application of the compound to the site of the bone defect. The required quantity of the composite may be applied after it has been mixed immediately prior to use with sterile water, sterile saline solution or a similar liquid, or else with the patient's own blood, in order to obtain a gelatinous paste that is dense but easily workable. The bone defect may have been created surgically or may be the natural result of a trauma or illness. The composite may be used in dentistry as well as orthopaedics. The following conditions are examples for possible applications in the field of dentistry: increase/reconstruction of tooth sockets, filling of defects following root-end resection, cystectomy, surgical removal of impacted teeth, filling of the tooth sockets following removal of a tooth in order to maintain the ridge, preparation of an implant bed, stabilization of immediate implants, bone dehiscence. In orthopaedics, examples for the use of the composite are maxillofacial surgery, joint reconstruction, repair of fractures, surgical orthopaedic procedures, spinal fusions.

The composite may be used together with one or more biologically active agents. Said agents are chosen from the following group: vitamins, mineral supplements, modulators of osteoblast or osteoclast functions, antimicrobial agents, antifungal agents, antibacterial agents, antiviral agents, antiparasitic agents, growth factors, angiogenic factors, anaesthetics, mucopolysaccharides, cells, proteins, enzymes, peptides.

Said antimicrobial agents are chosen from the following group: isoniazid, ethambutol, pyrazinamide, streptomycin, clofazimine, rifabutin, fluoroquinolones, ofloxacin, doxycycline, ampicillin, amphotericin B, ketoconazole, fluconazole, pyrimethamine, sulfadiazine, clindamycin, lincomycin, pentamidine, atovaquone, paromomycin, diclazuril, acyclovir, brivudine, trifluridine, foscamet, penicillin, gentamicin, ganciclovir, itraconazole, miconazole, Zn-pyrithione and silver salts.

Said growth factors are chosen from the following group: basic bone GF, fibroblast GF, acidic fibroblast GF, nerve GF, epidermal GF, insulin-like GF 1 and 2, platelet-derived GF, skeletal GF, tumour angiogenesis GF, vascular endothelial GF alpha and beta, interleukin-8, granulocyte-macrophage colony-stimulating factor, interleukin, interferon.

Said mucopolysaccharides are chosen from the following group: heparin, heparin sulphate, heparinoids, dermatan sulphate, pentosan polysulphate, chondroitin sulphate, hyaluronic acid, cellulose, agarose, chitin, dextran, carrageenin.

Said ‘proteins’ are chosen from the following group: collagen, fibronectin, laminin, elastin, osteopontin, osteonectin, bone sialoprotein, alpha-2HS-glycoprotein, bone Gla protein, matrix Gla protein, bone phosphoglycoprotein, bone phosphoprotein, bone proteoglycan, protolipids, bone morphogenetic protein, cartilage-inducing factor, proteins associated with cartilage, proteins associated with dentin, proteins associated with enamel.

Said cells are chosen from the following group: bone marrow stem cells, osteoblasts, osteoclasts, osteocytes, blood cells, epithelial cells, odontoblasts, ameloblasts, cementoblasts, neuronal cells.

The compositions created with the composite that is the subject of the present invention may also be used with various types of implants.

The following are some examples of the realization of the invention as described above:

### EXAMPLE 1

Preparation of the MgCHA

A 0.3 M aqueous solution of MgCl₂.6H₂O (48.4 g in 800 ml of water) is prepared at room temperature in a 2 l flask. 100 g of Ca(OH)₂ are dissolved in the same solution.

While stirring constantly with a mechanical stirrer, the resulting suspension is brought to a temperature of 40° C.±5° C.

Once the desired temperature is reached, a 1.3 M aqueous solution of H₃PO₄ (88.8 g in 600 ml of water) and a 0.8 M aqueous solution of NaHCO₃ (12.9 g in, 200 ml of water) are simultaneously added drop by drop; the dripping speed must be adjusted so that the addition is completed in 4 hours, while the stirring and the temperature are always kept constant.

Once the addition is completed, the mixture is stirred for another 2 hours with the temperature maintained at 40° C.±5° C. and is then left to rest for 24 hours and to return to room temperature. After 24 hours, the supernate is removed and the product is separated from the liquid residue through centrifugation.

The resulting hydroxyapatite is then washed three times with water by means of dispersion (using a magnetic stirrer) and is centrifuged.

The powder is air-dried in an oven at 80° C. for 12 hours and is stored at room temperature in a closed container. Yield: 120-140 g.

Characteristics of the Resulting Compound:

Appearance: white powder.

Plasma emission spectrometry (ICP-AES): Ca: 31.81%, Mg: 1.57%, P: 14.86% in weight (molar ratio (Ca⁺ Mg)/P equal to 1.79). The quantity of Mg is thus equal to 8.1% expressed as molar percentage with respect to the calcium.

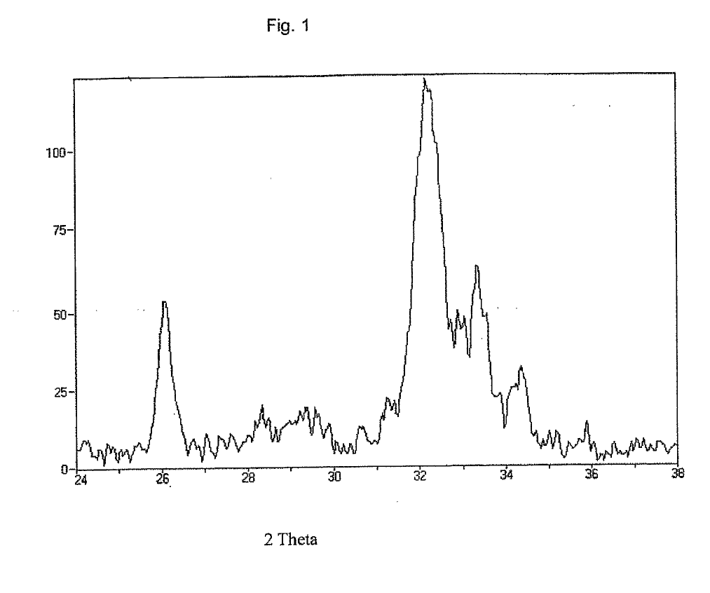

X-ray diffractometry (see FIG. 1): the diffraction spectrum under x-ray examination allows us to see the characteristic signs of apatites and to obtain information about the variation of some cell parameters (in particular axes (a) and (c)), which indicate the presence of both Mg²⁺ ions (substituting Ca²⁺ ions) and CO₃²⁻ ions inside the apatite's crystalline structure.

In fact, the spectrum shows that:

axis (a) of the cell experiences a reduction of 1.18% compared to that of apatite;

axis (b) of the cell experiences a reduction of 0.81% compared to that of apatite.

The presence of Mg²⁺ ions in place of the Ca²⁺ ions inside the apatite's structure leads to a reduction of the values of both axis a and axis c of the crystalline cell, which is due to a shorter ionic radius of the Mg²⁺.

As for the CO₃²⁻ group, its presence leads to a reduction of axis a and an increase of axis c in the case of phosphatic substitution (site B). In the case of a substitution of the hydroxyl group (site A), the variation is the other way around and of a different extent.

Therefore, the axis values calculated by us are the result of the influence of several substituents, which have synergetic and opposing effects on the above-mentioned axis values.

By combining the x-ray diffraction with FTIR and TG analysis, it becomes possible to quantify the extent of each different and single substitution.

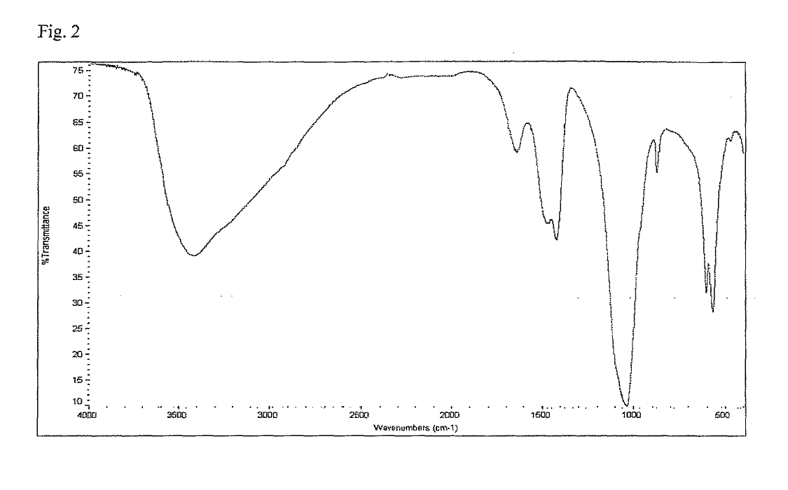

IR spectroscopy (see FIG. 2): The FTIR spectrum shows the signals relating to the groups PO₄³⁻, HPO₄²⁻, OH⁻ and CO₃²⁻.

The signal at 874 cm⁻¹, typical for the CO₃²⁻ group, provides information about the apatite's carbonation type. Through the deconvolution analysis of the carbonate peak it becomes possible to deduce that the powder's carbonation is predominantly of tape B with an A/B ratio of ≈0.75 (approx. 57% B and 43% A).

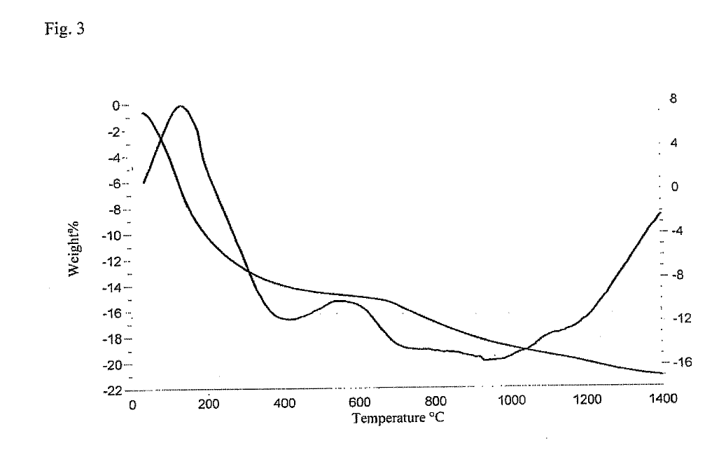

Thermogravimetric analysis (TG-DTA) (see FIG. 3): The thermal analysis provides qualitative and quantitative data about the product. At low temperatures, a first reduction of the weight is observed, which is due mainly to the loss of water and CO₂ adsorbed on the sample's surface and water occluded in the structure. A second weight loss at approx. 600-700° C. is due to the decarbonation process, while the dehydroxylation process begins at temperatures above approx. 900° C.

The analysis shows that the total carbonate content of the powder is approx. 6 wt %; this analysis does not allow for a distinction between site A and site B.

The reaction has been shown to be completely reproducible, as various tests have resulted in products with absolutely comparable characteristics and with molar ratios of (Ca⁺ Mg)/P=1.79±0.07.

### EXAMPLE 2

The process described in Example 1 is repeated using a smaller quantity of magnesium salt equal to 42.35 g of hexahydrated MgCl₂ dissolved in 800 ml (0.26 M).

The resulting compound is absolutely comparable to that described in Example 1 and presents the following values for the ICP analysis: Ca: 32.02%, Mg: 1.33%, P: 14.91% in weight (molar ratio (Ca+Mg)/P equal to 1.77). The quantity of Mg is thus equal to 6.8% expressed as molar percentage with respect to the calcium.

The other characterizing parameters can be superimposed on those obtained for the compound of Example 1.

### EXAMPLE 3

The process described in Example 1 is repeated using a smaller quantity of magnesium salt equal to 36.25 g of hexahydrated MgCl₂ dissolved in 800 ml (0.22 M).

The resulting compound is absolutely comparable to that described in Example 1 and presents the following values for the ICP analysis: Ca: 32.21%, Mg: 1.20%, P: 14.94% in weight (molar ratio (Ca⁺ Mg)/P equals 1.77). The quantity of Mg is thus equal to 6.1% expressed as molar percentage with respect to the calcium.

The other characterizing parameters can be superimposed on those obtained for the compound of Example 1.

### EXAMPLE 4

Preparation of the Composite

The MgCHA powder obtained in Example 1 is sifted to 150 μm. 50 g of a mixture of carbonate magnesium apatite powder and sodium alginate powder (Protanal LF 10/60 B.H. Schilling) with a weight ratio of 60/40 are placed in a mixer. The powders are initially homogenized in a dry state for approx. 10 seconds.

10 ml of absolute ethyl alcohol are added and the mixer is switched on for approx. 20 seconds. The mixer is stopped, and any preparation left on the walls or blades is removed with a spatula. This process is repeated 6-8 times until a total of 40 ml of alcohol has been added.

The resulting composite is dried in the oven at 60° C. for 10 minutes and the residual solvent is removed through evaporation with a freeze-drying process at −45° C. for 24 hours.

Characteristics of the Resulting Composite

Appearance: porous granular powder

ICP analysis: The commercial alginate used has a declared calcium content of <1.5%. The presence of this calcium affects the determined molar ratio Ca⁺ Mg/P, which was found to be 1.82±0.07.

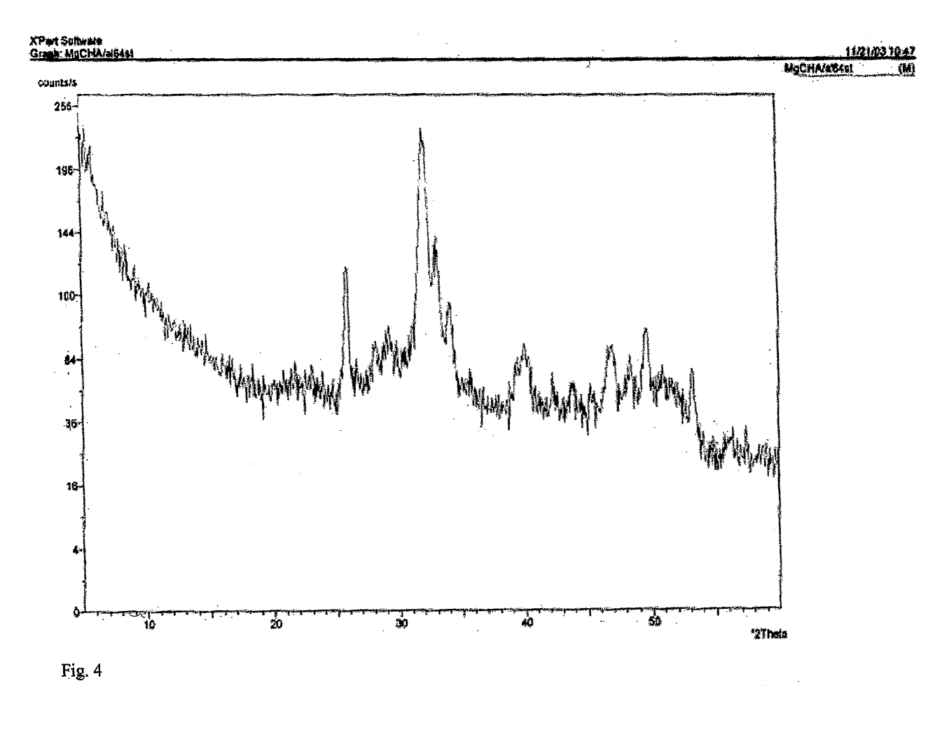

X-ray diffractometry (see FIG. 4): Analysis using x-ray diffractometry confirms that the inorganic phase was not compromised during the granulation process with alginate; this can be seen from the characteristic bands for 2-theta values between 25 and 40.

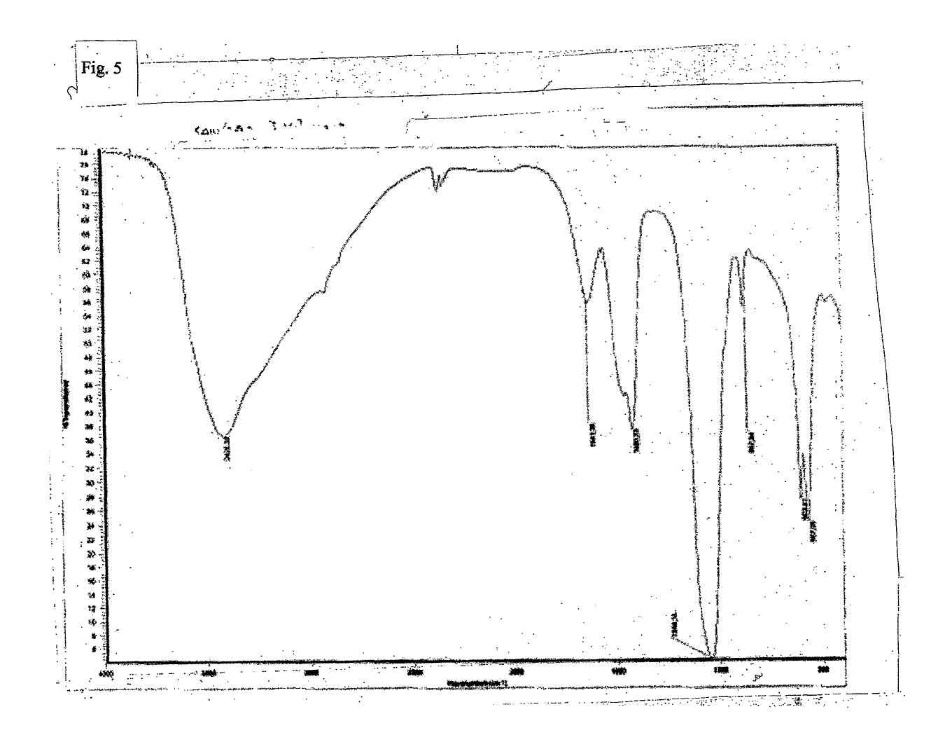

IR spectroscopy (see FIG. 5): Infrared spectroscopy as well shows the characteristic profile of the magnesium carbonate hydroxyapatite to be dominant; this profile is practically identical to the one obtained prior to the treatment with alginate (see FIG. 2).

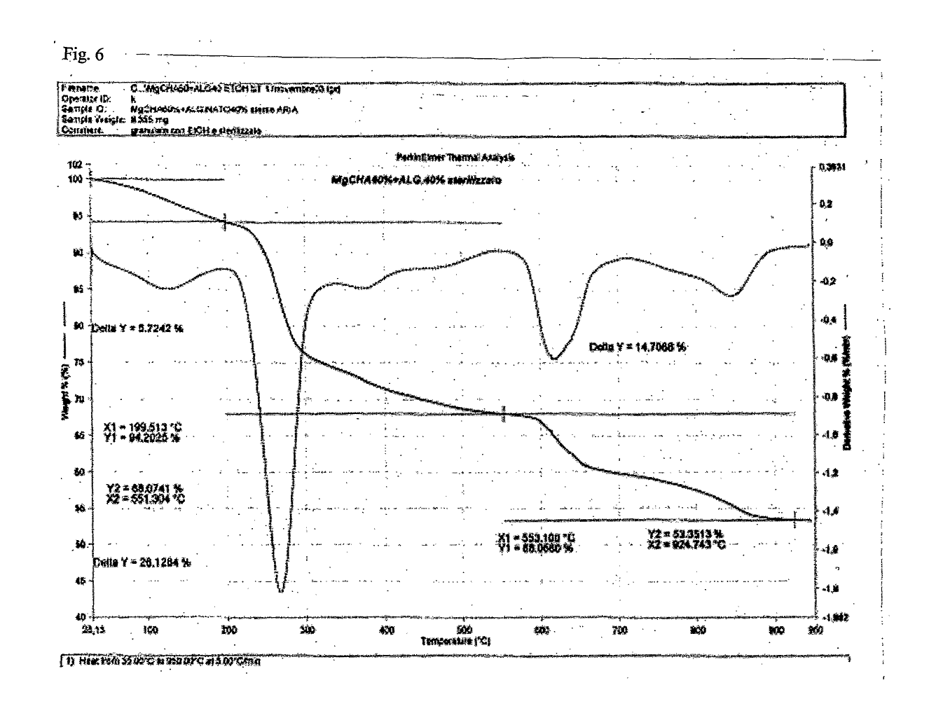

Thermogravimetric analysis (TG-DTA) (see FIG. 6): The profiles resulting from the thermal treatment show the weight decreases relating to the decomposition of both the organic and the inorganic phase.

At low temperatures of up to 500-550° C. there is a loss of CO2 and water adsorbed on the granulate and of water occluded in the structures of both the apatite and the alginate; around 600° C. the alginate decomposes; between 600 and 650° C. and between 800 and 900° C. we observe the decarbonation of the apatite and the combustion of the organic residues; finally, still around 800° C. the dehydroxylation takes place.

The resulting composite shows

stability of the inorganic component after the formation of the compound and

stability of the final granulate's weight in the presence of variations in the environmental humidity.

The composite material has a residual water content below 5 wt %.

Through sifting it is possible to obtain a fraction with a particle size between 250, and 500 micron.

### EXAMPLE 5

Humid preparation of the composite (this example does not result in a final composite material that is acceptable for the purpose of this invention)

50 g of a mixture with a weight ratio of 60/40 of the carbonate magnesium hydroxyapatite powder obtained in Example 1 and sodium alginate powder are placed in a mixer and are treated as in Example 4, with the exception that water is used in place of ethyl alcohol. The final granulate is dried in the oven at 60° C. for 30 minutes and is then dried out on a silica bed at room temperature.

X-ray analysis shows a profound alteration of the inorganic component. Moreover, a continuous change in the granulate's weight is observed over time due to absorption of humidity from the environment.

Pharmacological Activities

The product described in Example 4 is biocompatible, i.e. once implanted into the host, the material does not induce pathogenic reactions such a inflammations or tissue necrosis. The product's biocompatibility has been assessed in studies both in vitro and in vivo. In particular, in order to examine the biological reaction of cells to the selected compound, in vitro studies have been carried out to show any potential effects caused by the direct or indirect contact of the examined material with cell lines of various origins following short-term and medium-term exposure. The results show that the product is not cytotoxic. In vivo studies have shown that the preparation does not induce skin sensitisation reactions and thus further confirm the product's good biocompatibility.

The described product efficiently repairs bone defects following application of the necessary amount of product at the level of the existing cavity in the bone. For example, the examined product's capacity to repair a bone defect induced at the level of the lateral condyle of the femur (loss of substance) in rabbits was evaluated. The assessments showed that the product does not induce any inflammatory reactions or tissue necrosis significantly different from the control group. Moreover, the product has demonstrated excellent osteoconductive properties, absorption times suitable for the intended purpose and the capacity to speed up the physiological reaction of endogenous bone repair.

1. Magnesium carbonate hydroxyapatite (MgCHA) in which the percentage of carbonation falls within a range of 4 to 10 wt % and which contains a percentage of 5 to 15% of magnesium (expressed as molar percentage with respect to calcium).

2. The MgCHA according to claim 1, in which the molar percentage of magnesium with respect to calcium falls within a range of 6 to 8%.

3. The MgCHA according to claim 2, in which the carbonate is distributed at a rate of 40 to 45% at site A and 60 to 55% at site B.

4. The MgCHA according to claim 1, having a nanostructure with a low level of crystallinity, in which the degree of crystallinity is between 40 and 60%.

5. A composite consisting of a hydroxyapatite and an organic polymer, wherein the hydroxyapatite is an MgCHA according to claim 1, that the polymer is a natural polysaccharide and that these components are mixed according to a method that makes it possible to keep the MgCHA in a solid form.

6. A composite according to claim 5, in which the polysaccharide is a sodium alginate.

7. A composite according to claim 1, in which the MgCHA and the alginate are present in proportions from 50:50 to 80:20.

8. A composite according to claim 7, in which the ratio between the MgCHA and the alginate is 60:40.

9. A composite according to claim 5, obtained in the form of a porous granulate, which at the moment of therapeutic use is easily convertible to the form of a viscous paste through treatment with aqueous solutions.

10. A composite according to claim 5 obtained in a form chosen from the following: gel, malleable paste, malleable putty, sponge, particulate or pre-formed solid that can be moulded according to the application requirements.

11. Process for the synthesis of the MgCHA according to claim 1, in which
a) a phosphoric acid solution and a sodium bicarbonate solution are simultaneously added over a period of between 2 and 8 hours to a suspension of calcium hydroxide and magnesium salt in water at a temperature between 40 and 60° C.,
b) the resulting mixture is stirred for 1 to 6 hours at a temperature between 30 and 60° C. and is subsequently left to rest at room temperature for 10 to 48 hours,
c) the MgCHA is separated by centrifugation or filtration, washed and dried in an oven.

12. A synthesis process according to claim 11, in which the magnesium salt is hexahydrated magnesium chloride.

13. A synthesis process according to claim 11, in which the phosphoric acid and the sodium bicarbonate are added over a period of 3 to 5 hours.

14. A synthesis process according to claim 11, in which the reaction temperature is between 35 and 45° C.

15. A synthesis process according to claim 11, in which the mixture is left to rest at room temperature for a period of 20 to 28 hours.

16. Process for the preparation of the composite according to claim 5 in the form of a porous granulate, wherein an initial dry mixing of the MgCHA and polysaccharide polymer powders in proportions ranging from 50:50 to 80:20, preferably 60:40, and subsequently by mixing in the presence of a solvent on the basis of alcohol or ether and by the complete evaporation of the solvent.

17. A process according to claim 16, in which the solvent is ethyl alcohol or ethyl ether.

18. Compositions containing a composite material according to claim 5.

19. Compositions according to claim 18, in which said composite is in the form of a porous granulate that prior to therapeutic use can be transformed into a dense but easily workable gelatinous paste through treatment with an aqueous solution.

20. Compositions according to claim 18 for the treatment of patients with a loss of bone substance through application of the composite at the level of the bone defect.

21. Compositions according to claim 20, where the bone defect may occur in the fields of orthopaedics or dentistry and may be induced surgically or may occur naturally following a trauma or illness.

22. Compositions according to claim 21, where the bone defect to be treated relates to a dental treatment selected from the following group:
increase/reconstruction of tooth sockets, filling of defects following root-end resection, cystectomy, surgical removal of impacted teeth, filling of the tooth sockets following removal of a tooth in order to maintain the ridge, preparation of an implant bed, stabilization of immediate implants, bone dehiscence.

23. Compositions according to claim 21, where the bone defect to be treated relates to an orthopaedic treatment selected from the following group: maxillofacial surgery, joint reconstruction, repair of fractures, surgical orthopaedic procedures, spinal fusions.

24. Compositions according to claim 18, which together with the composite also contain one or more biologically active agents.

25. Process for the preparation of compositions for the treatment of bone defects in the field of dentistry selected from the following group: increase/reconstruction of tooth sockets, filling of defects following root-end resection, cystectomy, surgical removal of impacted teeth, filling of the tooth sockets following removal of a tooth in order to maintain the ridge, preparation of an implant bed, stabilization of immediate implants, bone dehiscence wherein the composites according to claim 5 are used.

26. Process for the preparation of compositions for the treatment of bone defects in the field of orthopaedics selected from the following group: maxillofacial surgery, joint reconstruction, repair of fractures, surgical orthopaedic procedures, spinal fusions wherein the composites according to claim 5 are used.

27. Process for the preparation of compositions to be used in combination with one or more biologically active agents wherein the composites according to claim 5 are used.

28. Process for the preparation of compositions to be used together with various types of implants wherein the composites according to claim 5 are used.
